(19) United States
(12) Patent Application Publication
(10) Pub. No.: US 2004/0133046 A1
(43) Pub. Date: Jul. 8, 2004

(54) Continuous process for preparing alkoxynitroarenes

(76) Inventors: Thomas K. Highsmith, North Ogden, UT (US); Harold E. Johnston, Brigham City, UT (US)

Correspondence Address: TRASK BRITT, P.O. BOX 2550, SALT LAKE CITY, UT, 84110, US

(21) Appl. No.: 10/338,767

(22) Filed: Jan. 7, 2003

(51) Int. Cl. C07C 205/00

(52) U.S. Cl. 568/584

(57) ABSTRACT

A continuous process for preparing alkoxynitroarenes, such as 2,4-dinitroanisole (DNAN) and 1-isopropoxy-2,4-dinitrobenzene, is provided. The continuous process of the present invention provides for the effective handling and manufacture of alkoxynitroarenes and permits the utilization of the continuous processing equipment already in place in a number of trinitrotoluene (TNT) manufacturing facilities. Thus, the present invention provides a continuous process for the large scale manufacture of TNT alternatives which does not require re-facilitization.

### BACKGROUND OF THE INVENTION

1. Field of the Invention

The present invention relates generally to processes for manufacturing nitroaromatic materials for use in high explosive compositions. More specifically, the present invention relates to a continuous process for manufacturing alternatives to trinitrotoluene (TNT) that permits the use of existing TNT manufacturing facilities.

2. State of the Art

Trinitrotoluene is an insensitive, high explosive often used in the manufacture of high-explosive ordnance. While very effective as an explosive compound, TNT has a number of drawbacks. For instance, TNT is rather difficult to produce with the nitration of the starting materials requiring multiple steps. Further, a large amount of waste is produced in the manufacture of TNT, much of which is both highly toxic and extremely stable. This poses an environmental concern as disposal of the waste produced is exceptionally difficult.

Accordingly, many in the explosives industry have begun to look at compounds which may be used as alternatives to TNT in the manufacture of high-explosive ordnance. Desirable alternatives would have a melting point around 80° C.-100° C., a low toxicity, would be easy to manufacture, and would produce no byproducts. One such alternative compound that has been extensively explored is Comp B (i.e., Composition B, generally comprised of 36% TNT, 63% RDX, 1% wax). While Comp B adequately addresses some of the drawbacks of TNT, it is an easily detonable compound. Thus, the manufacture of Comp B poses a high risk of adversely affecting process safety and occupational health. As such, less shock-sensitive alternatives to TNT have been, and continue to be, explored.

Potential alternatives to TNT and Comp B which are currently being investigated by those in the explosives industry are NTO (3-nitro-1,2,4-triazol-5-one or nitrotriazolone), fixed TNT (i.e., trinitrotoluene fixed with a polymer) and DNAN (2,4-dinitroanisole or 1-methoxy-2,4-dinitrobenzene). DNAN is a dinitroaromatic and is of particular interest as it is a lower cost, less toxic alternative to hazardous trinitroaromatics such as TNT. DNAN is in the PAX (Picatinny Arsenal Explosive) family of explosives and is a more insensitive (high) explosive to replace TNT. Conventional manufacturing processes for DNAN, however, have a number of drawbacks which render the large-scale manufacture thereof problematic.

The molecular formula of DNAN is C₇H₆N₃O₅. DNAN is generally found as a pale tan to buff-colored crystalline solid, although the impure material is often bright yellow due to contamination with nitrophenols. DNAN has two stable polymorphs: a low melting form, which has a melting point of approximately 89° C., and a high melting form, which has a melting point of approximately 94.6° C. The density of the commercial material is 1.341 g/cc. DNAN is only slightly soluble in water but highly soluble in most organic solvents including methyl and ethyl alcohols, as well as ethyl ether.

Nitration processes have been devised for the preparation of anisole derivatives such as DNAN. The direct nitration of anisole to produce 2,4,6-trinitroanisole (TNAN) was reported as early as 1849. This method, however, has not been employed industrially as the process is difficult to control and is prone to unexpected explosions. The isomeric, ortho- and para- mononitroanisoles have also been nitrated to yield both di- and trinitroanisoles. Unfortunately, these mononitroanisoles are not readily available starting materials nor are they readily prepared.

The direct nitration of DNAN is the currently preferred method for preparing TNAN. However, the nitration of anisole and anisole derivatives (e.g., DNAN) has a number of serious impediments to industrialization. For instance, all of the nitration processes involving anisole and its derivatives produce demethylated byproducts. The resulting nitrophenols are among the most undesirable of these byproducts. The prevalence of these impurities in any nitration process involving anisole greatly complicates the utilization of such chemical processes. These nitrophenols not only react with heavy metals to produce dangerously sensitive salts, but are also acutely toxic to humans. Consequently, the phenolic byproducts must be completely removed during the work-up from the reaction. The nitration of anisole and its derivatives also frequently produces undesirable, explosive byproducts, which can be controlled by dilution or cooling.

If larger quantities of these nitrophenol contaminants are present, removal of the phenolic byproducts greatly increases the wastewater production for the process and lengthens the purification process. For instance, the phenolic impurities often present in DNAN are best removed by washing the crude product thoroughly with water. Thus, considerable aqueous waste is produced in the preparation of high purity DNAN (i.e., approximately three gallons of aqueous waste per pound). Oftentimes, it is much more costly to adopt a process that incurs additional purification due to the disproportionately large increase in labor and materials involved in the purification of a less pure product, even if the raw materials are much less expensive.

Further, anisole is a relatively expensive raw material, costing approximately fourteen times as much as a similar grade of toluene. Accordingly, production of DNAN through nitration processes is oftentimes economically prohibitive.

In view of such difficulties in nitrating anisole and anisole derivatives, the most widely utilized method, at present, for the manufacture of DNAN involves the use of chlorobenzene rather than anisole as a starting material. In this mixed acid nitration process, chlorobenzene is nitrated at approximately 40° C. in the presence of nitric acid (HNO₃) and sulphuric acid (H₂SO₄) to yield 1-chloro-2,4-dinitrobenzene (CDB). Since the chlorine atom is only modestly deactivating to the ring, the nitration conditions are commensurately mild. The chlorine atom imparts chemical and physical properties similar to toluene to the starting chlorobenzene, as well as to the resulting products dinitrotolulene and dinitrochlorobenzene. This reaction is shown below as Reaction Scheme I.

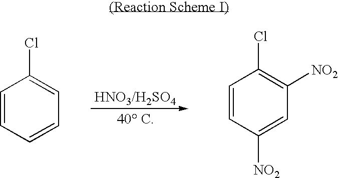

CDB is also commercially available at relatively low cost and in sufficient quantity.

DNAN may be manufactured from CDB by reacting it with methanol (CH₃OH) and an alkaline metal hydroxide (e.g., sodium hydroxide (NaOH)) under ambient conditions. As can be seen from Reaction Scheme II below, this reaction involves the displacement of chloride (Cl⁻) by the methoxide nucleophile (⁻OCH₃) and yields DNAN, alkaline metal chloride (e.g., NaCl) and water (H₂O). The methoxide nucleophile is slowly formed from the reaction between sodium hydroxide and methanol. The sodium hydroxide and methanol also produce hydroxide nucleophiles, which compete with the methoxide nucleophile to displace the chloride ion, reducing the yield and purity of the desired product. Alternatively, the methoxide nucleophile is formed from a reaction between sodium metal or sodium hydride with methanol. While formation of sodium methoxide from sodium hydroxide and methanol is slow, this reaction is preferred since sodium hydroxide is relatively cheap and readily available.

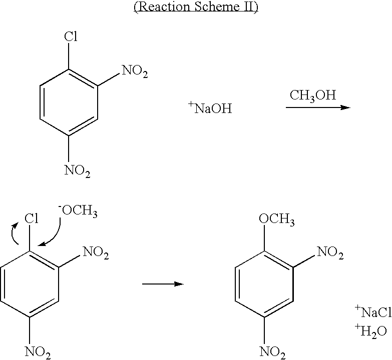

In the above-described process for the manufacture of DNAN (Reaction Scheme II), the reaction of alkaline metal hydroxide (e.g., NaOH) and methanol (CH₃OH) yields both methoxide (⁻OCH3) and hydroxide (⁻OH) nucleophiles. Thus, a certain number of hydroxide nucleophiles will displace the chlorine atom of CDB yielding 2,4-dinitrophenol, which is an undesirable, toxic byproduct (i.e., it is an uncoupler). While significantly less 2,4-dinitrophenol is produced (i.e., the yield of DNAN is approximately 80-85%), it is desirable to minimize the yield of this toxic byproduct as much as possible.

Further, the above-described process for the production of DNAN (Reaction Scheme II) is a batch process requiring large quantities of potentially toxic materials, e.g., high explosives and methanol, to be in process at one time. The large quantities of high explosives present in the batch process create the potential for a large explosion. The occupational and environmental safety implications of such a process are undesirable. In addition, a majority of existing TNT manufacturing facilities are continuous processing facilities. Accordingly, if conventional DNAN manufacturing processes are utilized to produce DNAN as a TNT alternative, such existing facilities cannot be utilized. Thus, in many instances, the expense involved in construction or purchase of a manufacturing facility may render the use of DNAN as a TNT alternative economically prohibitive.

In view of the above, the inventors have recognized that a continuous process for the effective handling and manufacture of alkoxynitroarenes, such as DNAN, which would permit the utilization of existing TNT manufacturing facilities would be advantageous.

### BRIEF SUMMARY OF THE INVENTION

The present invention relates to a continuous process for producing alkoxynitroarenes. The continuous process comprises substantially continuously supplying a stream of a nitroaromatic to a reaction vessel; substantially continuously supplying a stream of an alkaline metal hydroxide or an alkaline metal alkoxide to the reaction vessel; substantially continuously supplying a stream of one of methanol and isopropanol to the reaction vessel; substantially continuously mixing the nitroaromatic, the alkaline metal hydroxide or the alkaline metal alkoxide; stripping any unreacted of the one of methanol and isopropanol from the first mixture to produce a second mixture; subjecting the second mixture to a countercurrent wash with water to create a third mixture comprising water and product; and drying the third mixture to recover the product. The alkaline metal hydroxide is selected from the group consisting of lithium hydroxide, sodium hydroxide, potassium hydroxide, and rubidium hydroxide. The alkaline metal alkoxide is an alkaline metal isopropoxide selected from the group consisting of lithium isopropoxide, sodium isopropoxide, potassium isopropoxide, and rubidium isopropoxide.

This continuous process provides for the effective handling and manufacture of alkoxynitroarenes and permits the utilization of the continuous processing equipment already in place in a number of TNT manufacturing facilities. Thus, the present invention provides a continuous process for the large scale manufacture of TNT alternatives which does not require re-facilitization. Currently, explosive factories in the United States use continuous processes (e.g., Holston Army Ammunition Plant for RDX and HMX, as well as past plants which manufacture nitroguanidine, TNT, etc.) and, therefore, the continuous processing equipment in these facilities may be used in the present invention.

In a further embodiment the present invention relates to a continuous process for producing 1-chloro-2,4-dinitroanisole. The continuous process comprises substantially continuously supplying a stream of 1-chloro-2,4-dinitrobenzene to a reaction vessel; substantially continuously supplying a stream of methanol to the reaction vessel; substantially continuously supplying a stream of an alkaline metal hydroxide to the reaction vessel; substantially continuously mixing the 1-chloro-2,4-dinitrobenzene, methanol and alkaline metal hydroxide in the reaction vessel to produce a first mixture; stripping any unreacted methanol from the first mixture to product a second mixture; subjecting the second mixture to a countercurrent continuous wash with water to produce a third mixture comprising water and 1-chloro-2,4-dinitroanisole; and drying the third mixture to recover the 1-chloro-2,4-dinitroanisole. The alkaline metal hydroxide may be selected from the group consisting of lithium hydroxide, sodium hydroxide, potassium hydroxide, and rubidium hydroxide. It is currently preferred that the alkaline metal hydroxide is selected from the group consisting of lithium hydroxide, sodium hydroxide and potassium hydroxide. It is currently more preferred that the alkaline metal hydroxide comprises sodium hydroxide.

Still further, the present invention relates to a continuous process for preparing alkoxynitroarenes, such as 1-isopropoxy-2,4-dinitrobenzene or 1-chloro-2,4-dinitroanisole. The alkoxynitroarenes comprise from one to four carbons that are either straight chains or branched. To prepare 1-isopropoxy-2,4-dinitrobenzene, the continuous process comprises substantially continuously supplying a stream of 1-chloro-2,4-dinitrobenzene to a reaction vessel; substantially continuously supplying a stream of isopropanol to the reaction vessel; substantially continuously supplying a stream of an alkaline metal alkoxide to the reaction vessel; substantially continuously mixing the 1-chloro-2,4-dinitrobenzene, isopropanol and alkaline metal alkoxide in the reaction vessel to produce a first mixture; stripping any unreacted isopropanol from the first mixture to produce a second mixture; subjecting the second mixture to a countercurrent wash with water to produce a third mixture comprising water and 1-isopropoxy-2,4-dinitrobenzene; and drying the third mixture to recover the 1-isopropoxy-2,4-dinitrobenzene. The alkaline metal alkoxide may be selected from the group consisting of lithium isopropoxide, sodium isopropoxide, potassium isopropoxide, and rubidium isopropoxide. It is currently preferred that the alkaline metal alkoxide is selected from the group consisting of lithium isopropoxide, sodium isopropoxide and potassium isopropoxide. It is currently more preferred that the alkaline metal alkoxide comprises sodium isopropoxide.

Additional aspects of the invention, together with the advantages and novel features appurtenant thereto, will be set forth in the description which follows and will also become readily apparent to those of ordinary skill in the art upon examination of the following and from the practice of the invention.

### BRIEF DESCRIPTION OF THE DRAWINGS

In the accompanying drawings which form a part of the specification and are to be read in conjunction therewith:

FIG. 1 is a ChemCad model of a continuous TNT manufacturing system that is present at a number of TNT manufacturing facilities and may be used to enable the continuous production of alkoxynitroarenes such as 2,4-dinitroanisole (DNAN) and 1-isopropoxy-2,4-dinitrobenzene according to the present invention;

FIG. 2 is a ChemCad model of the pressure/temperature relationship of the process for preparing DNAN at elevated pressure and temperature as described in Example III hereof;

FIG. 3 illustrates the results of Proton Nuclear Magnetic Resonance Spectroscopy (NMR) performed on the product of a process for preparing DNAN according to Example III hereof;

FIG. 4 illustrates the results of Fourier Transform Infrared Spectroscopy (FTIR) performed on the product of a process for preparing DNAN according to Example III hereof;

FIG. 5 is a graph illustrating a Gas Chromatography/Mass Spectrometry (GC/MS) analysis of a process for preparing 1-isopropoxy-2,4-dinitrobenzene under ambient pressure and temperature conditions; and

FIG. 6 is a graph illustrating a GC/MS analysis of a process for preparing 1-isopropoxy-2,4-dinitrobenzene under elevated pressure and temperature conditions.

### DETAILED DESCRIPTION OF THE INVENTION

The present invention is directed to a continuous process for preparing alkoxynitroarenes. In particular, the present invention is directed to a continuous process for preparing alkoxynitroarenes, such as 2,4-dinitroanisole, that may be used as alternatives to trinitrotoluene (TNT) in high-explosive ordnance. The particular embodiments described herein are intended in all respects to be illustrative rather than restrictive. Other and further embodiments will become apparent to those of ordinary skill in the art to which the present invention pertains without departing from its scope.

The present invention provides a continuous process for the rapid, high yield preparation of alkoxynitroarenes, such as 2,4-dinitroanisole (DNAN) and 1-isopropoxy-2,4-dinitrobenzene. The alkoxynitroarenes comprise from one to four carbons that are either straight chains or branched. While the description and examples herein focus on the preparation of DNAN and 1-isopropoxy-2,4-dinitrobenzene, it will be understood and appreciated by those of ordinary skill in the art that the processes so described are equally applicable to other alkoxynitroarenes, the preparation of which does not conventionally involve the use of continuous processing.

The inventors have discovered that manufacturing alkoxynitroarenes such as DNAN cheaply, quickly and safely may be more feasible if continuous rather than batch processes are utilized. In continuous processing, a steady stream of starting materials is supplied into a reaction vessel and a steady stream of product is produced out of the reaction vessel. Reaction of the starting materials must occur within the time frame the materials are in the reaction vessel. During the process, a wash system may be used. The wash system is preferably a countercurrent wash system. However, it is understood that the wash system is not limited to a countercurrent wash system and may be any wash system, such as a co-current, a batch, a semi-batch, or a pot wash system.

Continuous processing is advantageous for explosives because increased thermal treatment may cause increased decomposition. Further, a majority of TNT manufacturing facilities use continuous processing, wherein toluene and mixed acids (e.g., sulfuric and nitric acids) are supplied into a reaction vessel and TNT, spent acid and water are produced out of the reaction vessel. Thus, a continuous process for manufacturing alternatives to TNT, e.g., DNAN, may permit the use of existing TNT facilities to produce such lower cost, less toxic alternatives. As used herein, the terms “continuous” and “substantially continuous” (as well as any variations thereof) are used to distinguish the described processes from batch processes wherein finite quantities (rather than ratios) of constituents are processed. Thus, the terms “continuous” and “substantially continuous” (and all variations thereof) refer to processes wherein constituents are provided and processed as a flow stream. The terms “continuous” and “substantially continuous” (and all variations thereof) do not exclude or preclude processes wherein one or more constituents or process steps are briefly stopped or interrupted.

The following are examples of continuous processing conditions under which DNAN and 1-isopropoxy-2,4-dinitrobenzene may be produced. While the examples are clearly within the scope of the invention, these examples are merely illustrative and are not meant to limit the scope of the present invention in any way.

### EXAMPLES

### Example I

### Continuous Preparation of DNAN

The reaction of 1-chloro-2,4-dinitrobenzene (CDB) with methanol (CH₃OH) and an alkaline metal hydroxide (e.g., sodium hydroxide (NaOH)) to produce DNAN is particularly suited for continuous processing as the reaction proceeds to a high degree of completion in a short period of time (i.e., less than five minutes). The continuous process herein described would be carried out under above-ambient pressure and temperature conditions. If desired, the continuous process herein described may be carried out with ambient temperature and pressure. However, in such case, the solution vapors produced from the alcohol solvent (CH₃OH) in the continuous manufacturing process should be vacuumed off or the temperature thereof increased (or both) for appropriate disposal or recycling. When utilizing existing TNT manufacturing facilities, this would require the installation of condensers on the reactors which is a significant facility modification. However, if the pressure and temperature are increased as described in the present example, the solution vapors of the alcohol solvent may be more readily flashed off without the need to install condensers on the reactors. Thus, the increased pressure and temperature conditions, while not necessary, would provide an engineering benefit when utilizing existing TNT manufacturing facilities.

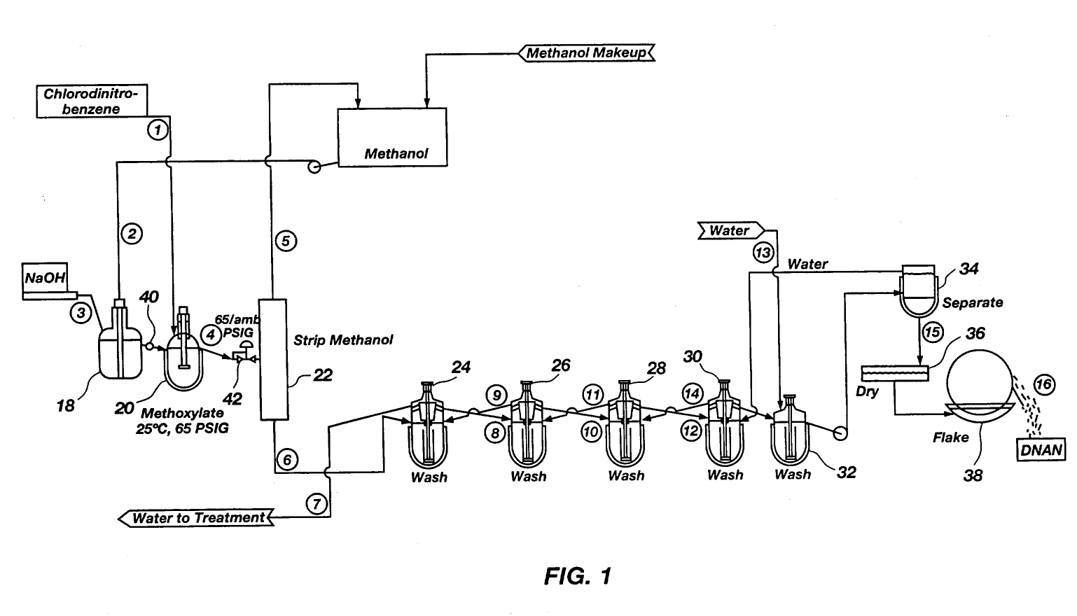

FIG. 1 depicts a continuous implementation that would take advantage of existing countercurrent washing and flaking facilities located at most, if not all, existing TNT manufacturing facilities worldwide. The continuous processing equipment illustrated in FIG. 1 includes a dissolution chamber 18, a stirred pressure reactor 20, a methanol-stripping column 22, a plurality of countercurrent wash vessels 24, 26, 28, 30, a wash chamber 32, a separation chamber 34, a drying station 36 and a flaking station 38. The countercurrent wash system need not be comprised of a plurality of wash vessels as illustrated but may be any countercurrent wash system known in the art (i.e., any system wherein water is passed countercurrent to the product). For instance, the countercurrent wash system may be a single extractor column or the like.

To produce DNAN utilizing the continuous processing equipment of FIG. 1, solid alkaline metal hydroxide (e.g., NaOH) would be continuously screw fed into the dissolution chamber 18 wherein it would be dissolved in a continuous stream of methanol which would also be flowing into the dissolution chamber 18. The stream of alkaline metal hydroxide is designated in FIG. 1 as stream 3 and the stream of methanol is designated as stream 2. The temperature of each stream would be approximately 21° C. and the pressure would be approximately 14.7 psig.

The dissolution chamber 18 would be a stirred and cooled tank for the dissolution of the alkaline metal hydroxide in methanol and would feed a pressure boost pump 40. The pump 40 would elevate the pressure of the combined stream exiting the dissolution chamber 18 to approximately 65 psig and feed such stream into the stirred pressure reactor 20. A separate stream of 1-chloro-2,4-dinitrobenzene (stream 1) would be pumped into the stirred pressure reactor 20 wherein the methoxylation of chlorodinitrobenze would take place. The pressure in the pressure reactor 20 would be maintained at approximately 65 psig and the temperature would be raised to approximately 125° C.

The outflow (stream 4) of the stirred pressure reactor 20 would flow through a pressure let down valve 42 directly to a methanol-stripping column 22 which would recover the surplus methanol for reuse (stream 5). Stream 4 would be maintained at a pressure of approximately 65 psig and a temperature of approximately 125° C. and would be comprised of methanol, water, alkaline metal hydroxide, alkaline metal chloride, DNAN and dinitrophenol.

Out of the methanol-stripping column 22, a stream (stream 6) of melted DNAN and byproducts would flow into a countercurrent washing and flaking system in which the byproducts would be efficiently washed from the immiscible DNAN melt phase with hot water. Stream 6 would be comprised of melted DNAN, water, alkaline metal hydroxide, alkaline metal chloride, dinitrophenol and a significantly lesser amount of methanol than was present in stream 4, as most of the methanol would have been stripped from the stream and recovered for reuse (stream 5).

As the stream flows through the countercurrent wash system, hot water (approximately 85° C.) would be supplied to the countercurrent wash vessels 24, 26, 28, 30 in a direction countercurrent to the product flow. A waste stream (stream 7) containing methanol, water, alkaline metal hydroxide, alkaline metal chloride, dinitrophenol and a small amount of DNAN would be washed out of the product stream through this process. While a small amount of the desired product (i.e., DNAN) would likely be lost, dinitrophenol is more soluble in water than DNAN and, thus, significantly more of this unwanted toxic byproduct would be washed from the product stream than desired DNAN lost. The exchange of the various streams through the serially aligned countercurrent wash vessels is shown in FIG. 1 as streams 8 through 12 and 14. The final washed product stream would flow into the wash chamber 32 wherein a final addition of hot water (stream 13) would be added.

The product would then be fed into the separation chamber 34 wherein the remaining water would be separated from the product stream. The product stream (stream 15) would then flow into the drying station 36 where any remaining water would be evaporated therefrom. Stream 15 would still be at an elevated temperature (i.e., approximately 85° C.) and at a pressure of approximately 14.7 psig. The stream would be comprised primarily of DNAN although trace amounts of methanol, water, alkaline metal hydroxide, alkaline metal chloride and dinitrophenol would also likely be present. The dried product would subsequently be flaked at the flaking station 38 to yield the desired DNAN product.

DNAN produced according to the described continuous process would permit the large scale manufacture thereof utilizing equipment already present in a number of TNT manufacturing facilities. Further, the increased pressure applied to the manufacturing process would enable control of the alcohol solvent vapors without significant modification to the TNT manufacturing facility.

It will be understood and appreciated by those of ordinary skill in the art that it would be desirable to produce as little solution vapors from the alcohol solvent as possible in the above-described reaction. This is because less waste would be produced which would have to be disposed of and because less product would be utilized, making the manufacture of DNAN more efficient. Due to the solubility limit, the minimum starting materials which may be used (i.e., the most concentrated the OH may be) is approximately 6 ml of methanol per gram of sodium hydroxide under ambient conditions. It is understood that at increased pressures and temperatures, the solubility limit is increased accordingly. Thus, it is desired to stay within this product proportion in carrying out the above-described continuous process so that less starting materials may be utilized and less waste may be produced.

Further, the inventors have found that the rate of reaction is controlled, at least in part, by the ratio of alkoxide (i.e., the conjugate base of the alkaline metal hydroxide) to the nitroaromatic starting material. As this ratio approaches 1.0 or less, the rate of reaction slows dramatically. Thus, it is also desirable to carry out the above-described reaction with an alkoxide to nitroaromatic ratio greater than 1.0.

### Example II

### Continuous Preparation of DNAN

A continuous implementation according to the reaction conditions described in Example I was conducted at a ratio of alkoxide to nitroaromatic (i.e., the conjugate base of NaOH (HO⁻) to CDB) of 1.2:1. That is, the reaction was run with a 20% excess of alkoxide. At 55° C., it was found that the kinetics of the reaction were as anticipated and the reaction proceeded to completion in less than one minute.

### Example III

### Preparation of DNAN at Elevated Pressure and Temperature Under Batch Processing Conditions

As previously described, when the ratio of alkoxide (i.e., the conjugate base of the alkaline metal hydroxide) to the nitroaromatic starting material (e.g., CDB) was very close to or less than 1.0, the rate of reaction was found to slow dramatically as the reaction progressed toward completion. While each reaction preferably begins with an excess of alkoxide, once a large portion of the particles have reacted with one another, the ratio of alkoxide to nitroaromatic decreases to 1.0 or less. Thus, the effect of increased pressure and temperature on accelerating the reaction through the final phases of completion was explored.

This example was carried out in a Parr high-pressure reactor having a reactor volume of 1 liter. The reactor lid was fitted with a stirrer, a thermocouple, a 1400-psi rupture disk and a vent valve. The reaction was carried out behind adequate shielding.

A solution of 20.2 grams, 100 mmol 1-chloro-2,4-dinitrobenzene (CDB) was prepared in 125 ml of reagent grade methanol. To this solution, 8.8 grams, 110 mmol of a 50% sodium hydroxide solution was added. The reaction mixture thus prepared was transferred to the Parr high-pressure reactor with the aid of a small amount of methanol. The reactor top was fitted and the pressure ring was fitted and torqued down. The bottom three inches of the reactor was then placed in a sand-filled, electrically preheated mantle. Table I shows the temperature/pressure correlation for the reaction.

TABLE I
Pressure/Temperature Correlation

| Time | Temperature | Pressure |
|---|---|---|
| (minutes) | (° F.) | (psi) |
| 0 | 70 | — |
| 10 | 228 | 40 |
| 20 | 243 | 55 |
| 30 | 254 | 60 |
| 37 | 257 | 60 |
| 40 | 255 | 60 |

After the reaction had been held at about 255° F. for approximately ten minutes, the reactor was removed from the heated mantle and placed in an ice bath. Once the pressure gauge of the reactor read 0 psi, the vent was opened to release any slight residual overpressure. The reactor lid was subsequently removed. The reactor contained a bright yellow slurry. A sample of the liquid was submitted to thin layer chromotographic analysis on silica gel using 20% ethyl acetate hexane. The chromatogram (not shown) indicated the complete absence of starting 1-chloro-2,4-dinitrobenzene and only a trace amount of 2,4-dinitrophenol byproduct.

The contents of the reactor were then transferred to a beaker and diluted with an approximately equal volume of water. The slurry was then filtered and the solids washed three times with 100 ml of distilled water. The remaining solids were subsequently air dried for approximately twenty-four hours. The product consisted of 17.7 grams of tan-colored needles having a melting point of between 83.5 and 85.8° C.

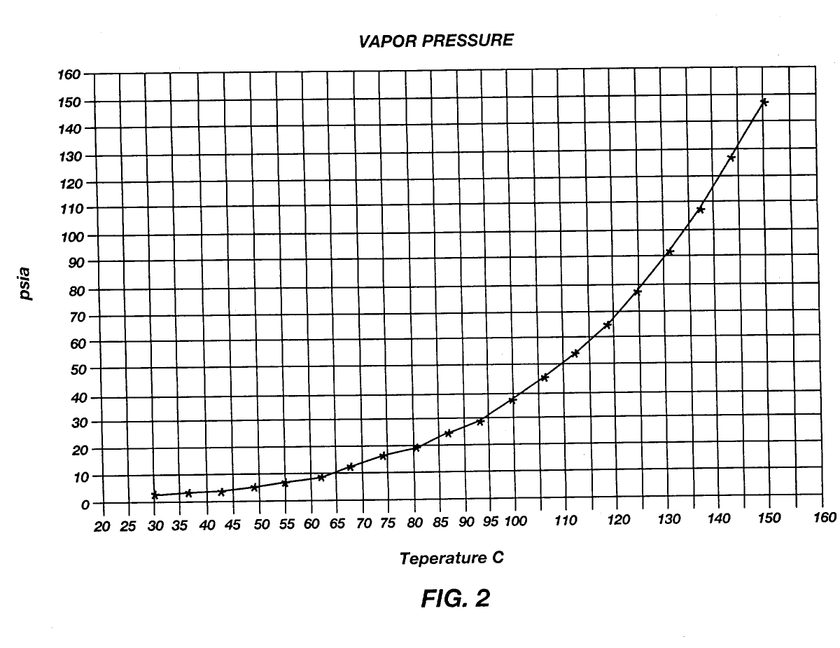

A ChemCad model of the pressure/temperature relationship for the reaction was generated (FIG. 2). As is evident, the reaction behaved largely as predicted, i.e., as the pressure in the reactor was increased, the temperature increased as well, increasing the rate of reaction.

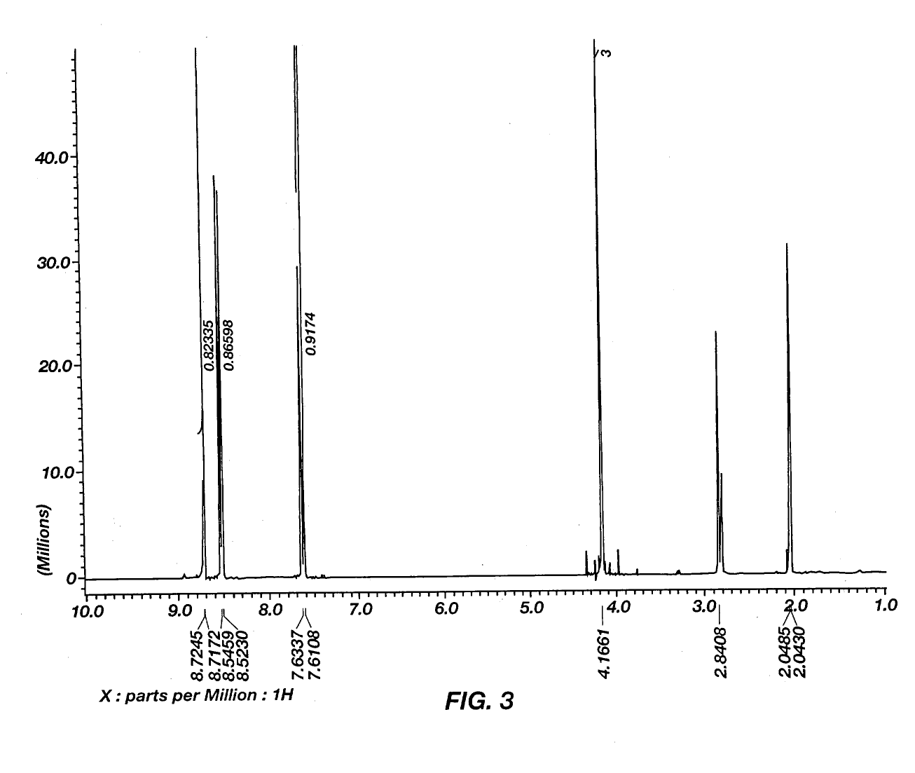

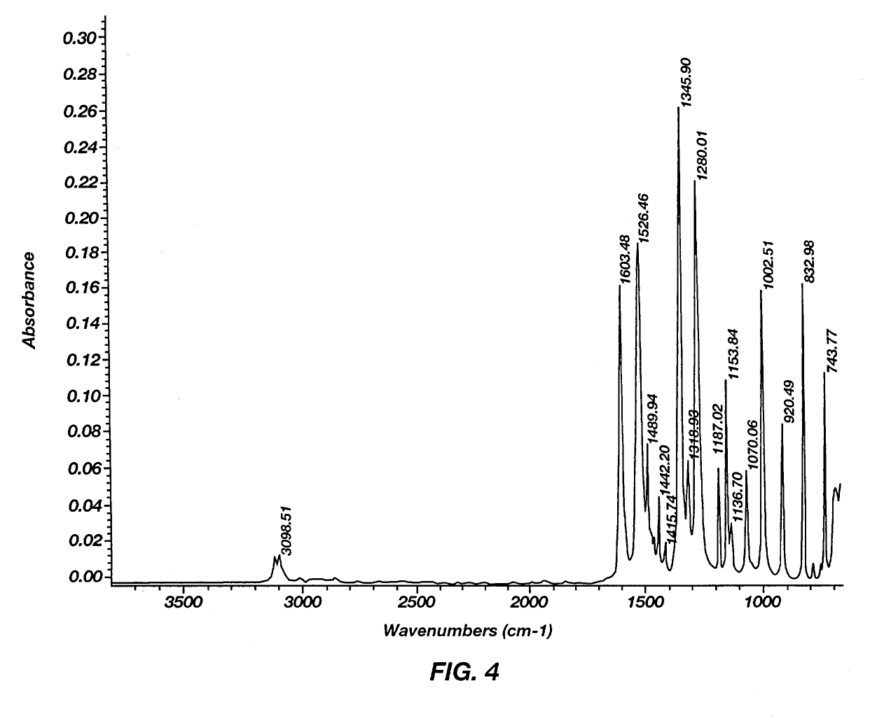

Proton Nuclear Magnetic Resonance Spectroscopy (NMR) was performed on the product in d6 acetone. The NMR indicated that the material was greater than 99.8% pure DNAN, as evidenced by the lack of any resonances other than the desired product (FIG. 3). Fourier Transform Infrared Spectroscopy (FTIR) was also performed which confirmed the presence of the methoxy and nitro functional groupings on the ring (FIG. 4).

As previously mentioned, under conventional reaction conditions, the yield of DNAN is approximately 80-85%. However, under the increased temperature and pressure conditions of the process of the present example, the yield of pure DNAN was increased to greater than 99.8%. Considering the toxicity of the 2,4-dinitrophenol that comprises at least a portion of the remaining reaction products, this higher yield, less waste-producing alternative would be highly desirable. However, a product having the demonstrated increased purity was unexpected.

Accordingly, to test the effect that increased pressure and temperature conditions have on the purity of the resultant product, 1-isopropoxy-2,4-dinitrobenzene was prepared under ambient pressure conditions and increased pressure conditions, such experiments shown respectively as Examples IV and V below.

### Example IV

### Preparation of 1-isopropoxy-2,4-dinitrobenzene Under Ambient Conditions

Preparation of the alkoxynitroarene 1-isopropoxy-2,4-dinitrobenzene was chosen as the branched, secondary alkoxide of the sodium isopropoxide starting material is more hindered than the NaO⁻ utilized in the preparation of DNAN. Thus, it was hypothesized, consistent with chemical theory, that any effect of the reaction conditions on the rate of reaction would be amplified.

A stock solution of sodium isopropoxide was prepared by adding 6.0 grams of sodium hydride (0.25 moles) to 400 ml of reagent grade isopropanol. The stock solution was prepared under a stream of nitrogen gas with constant agitation. The slurry became difficult to stir near the end of the hydride addition. After all of the hydride had been added, heat was applied to cause the reaction to completely proceed into solution. This solution was then set aside.

One hundred mg of reagent grade isopropanol was placed in a 500 ml, three-necked round bottom flask equipped with a magnetic stir bar, argon inlet, condenser and thermocouple. To this solvent, 26.0 grams of 1-chloro-2,4-dinitrobenzene (CDB) was dissolved with gentle heating. Complete solution was observed at 80° C. To this solution, 200 ml, 0.97 equivalents of the pre-prepared sodium isopropoxide solution in isopropanol was added. The isopropoxide solution was added as rapidly as possible such that the contents of the reaction remained in the flask. Three ml samples were taken as soon as the combination had been made and subsequently at periodic intervals thereafter. The intervals were frequent at the beginning of the reaction but were less frequent at later times during the reaction. The sampling intervals are indicated on the GC/MS. A portion of each sample was subjected directly for Gas Chromatography/Mass Spectrometry (GC/MS) analysis. The samples were evaporated, dissolved in deuterated chloroform and subjected to Proton Nuclear Magnetic Resonance Spectroscopy (NMR).

After refluxing a total of 22 hours, the contents of the reactor were poured onto 1 liter of ice and water. An additional 1 liter of water was then added to decrease the solubility of the product. The yellow brown solid was collected on a glass frit and washed three times with distilled water. After air drying, 15.0 grams of solid remained. Proton NMR revealed the solid to be 88% desired 1-isopropoxy-2,4-dinitrobenzene and 12% unconverted 1-chloro-2,4-dinitrobenzene.

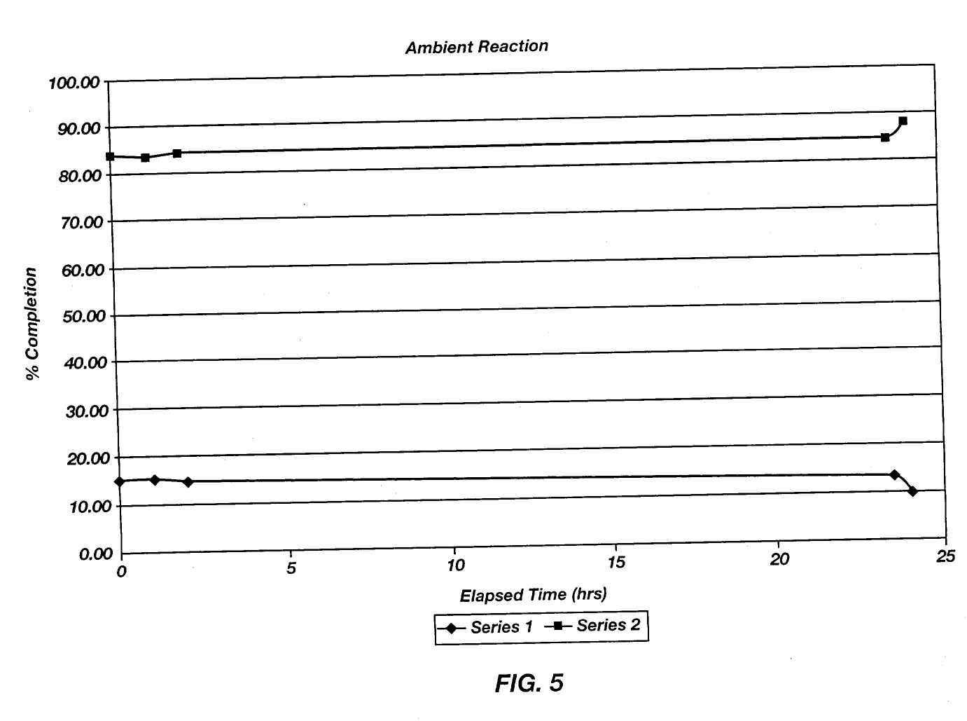

The GC/MS which resulted was compiled and plotted over time with respect to percent conversion. The data are summarized in FIG. 5. This data was verified by proton NMR as well (not shown). As is evident, the percent conversion was approximately 84% immediately upon mixing of the reagents. The additional approximately 4% conversion after about twenty-four hours may be attributed to decreased reaction time when the alkoxide (i.e., the conjugate base of sodium isopropoxide) to nitroaromatic (i.e., CDB) ratio reached approximately 1.0.

### Example V

### Preparation of 1-isopropoxy-2,4-dinitrobenzene Under Increased Pressure Conditions

A stock solution of sodium isopropoxide was prepared by adding 6.0 grams of sodium hydride (0.25 moles) to 400 ml of reagent grade isopropanol. The stock solution was prepared under a stream of nitrogen gas with constant agitation. The slurry became difficult to stir near the end of the hydride addition. After all of the hydride had been added, heat was applied to cause the reaction to completely proceed into solution. This solution was then set aside.

Twenty-six grams (0.129 moles) of 1-chloro-2,4-dinitrobenzene was dissolved in 100 grams of isopropanol. This solution was subsequently placed in a 300 ml capacity Parr high pressure reactor equipped with a pressure gauge, a rupture disk, a pressure relief valve, a mechanical stirrer and a bottom sampling tube with high pressure valve. To this pale yellow homogenous solution was added, in one portion, 200 ml of the pre-prepared isopropoxide solution (3.0 grams, 0.125 mole, 0.97 mole equivalents). Three ml samples were taken as soon as the reagents had been combined and subsequently at periodic intervals thereafter as indicated on the GC/MS plot. A portion of each sample was subjected directly for Gas Chromatography/Mass Spectrometry (GC/MS) analysis. The samples were evaporated, dissolved in deuterated chloroform and subjected to Proton Nuclear Magnetic Resonance Spectroscopy (NMR).

The reaction temperature was increased to 172° C. over the course of one hour. The pressure increased to 180 psi. The reaction was allowed to remain under these conditions for another half hour after which the heat was removed. The reaction was subsequently allowed to cool overnight. The contents of the reactor were subsequently poured into 1 liter of water and the solids collected on a glass frit. The solids were washed with three 100 ml portions of distilled water. After air drying, the yellowish-green solid weighed 18.1 grams. Proton NMR revealed a purity of 95%. The samples pulled during the run accounted for approximately 5 grams of starting material resulting in a yield of approximately 90%.

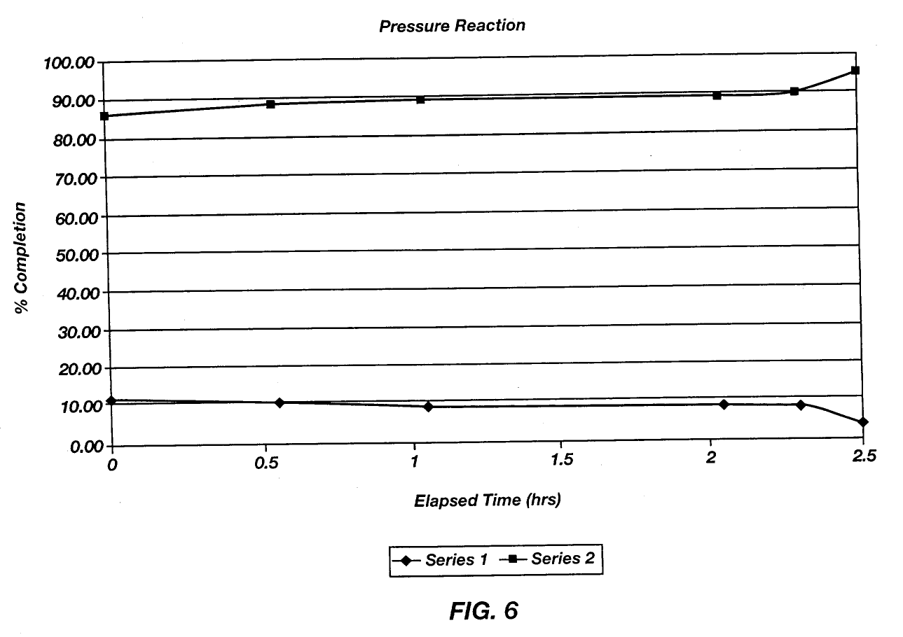

The Gas Chromatography/Mass Spectrometry (GC/MS) analysis which resulted was compiled and plotted over time with respect to percent conversion. The data are summarized in FIG. 6. This data was verified by NMR as well (not shown). As is evident, the percent conversion was approximately 87% immediately upon mixing of the reagents. The additional approximately 8% conversion over the subsequent approximately 2.5 hours may be attributed to decreased reaction time when the alkoxide (i.e., the conjugate base of sodium isopropoxide) to nitroaromatic (i.e., CDB) ratio reached approximately 1.0. When compared to the reaction carried out under ambient pressure conditions, it is evident that the remaining conversion occurred much more quickly in the increased temperature and pressure reaction (i.e., in approximately 2.5 hours rather than twenty-four hours).

A comparison of the percent conversion between examples IV and V revealed that there was an approximately 2% overall increase in the desired product yield when the reaction was carried out under increased pressure conditions. While not being held to any one theory, the inventors have, at present, attributed this increased product yield to the kinetics of the reaction including the increased rate of reaction caused by the addition of pressure when the ratio of alkoxide to nitroaromatic reached 1.0 or less.

In summary, the present invention provides a continuous process for the effective handling and manufacture of alkoxynitroarenes which permits the utilization of the continuous processing equipment already in place in most, if not all, existing TNT manufacturing facilities worldwide. Thus, the present invention provides a continuous process for the large scale manufacture of TNT alternatives which does not require re-facilitization.

The present invention has been described in relation to particular embodiments which are intended in all respects to be illustrative rather than restrictive. Other and further embodiments will become apparent to those skilled in the art to which the present invention pertains without departing from its scope.

From the foregoing, it will be seen that this invention is one well adapted to attain all the ends and aspects hereinabove set forth, together with other advantages which are obvious and which are inherent to the described process. It will be understood and appreciated that certain features and subcombinations are of utility and may be employed without reference to other features and subcombinations. This is contemplated by and is within the scope of the claims. Since many possible embodiments may be made of the invention without departing from the scope hereof, it is to be understood that all matter herein set forth is to be interpreted as illustrative and not in a limiting sense.

What is claimed is:

1. A continuous process for preparing alkoxynitroarenes, comprising:
substantially continuously supplying a stream of a nitroaromatic to a reaction vessel;
substantially continuously supplying a stream of an alkaline metal hydroxide or an alkaline metal alkoxide to the reaction vessel;
substantially continuously supplying a stream of one of methanol and isopropanol to the reaction vessel;
substantially continuously mixing the nitroaromatic, the alkaline metal hydroxide or the alkaline metal alkoxide, and the one of methanol and isopropanol in the reaction vessel to produce a first mixture;
stripping any unreacted of the one of methanol and isopropanol from the first mixture to produce a second mixture;
subjecting the second mixture to a countercurrent wash with water to create a third mixture comprising water and product; and
drying the third mixture to recover the product.

2. The continuous process of claim 1, wherein substantially continuously supplying a stream of a nitroaromatic to the reaction vessel comprises substantially continuously supplying a stream of 1-chloro-2,4-dinitrobenzene to the reaction vessel.

3. The continuous process of claim 1, wherein substantially continuously supplying a stream of an alkaline metal hydroxide or an alkaline metal alkoxide to the reaction vessel comprises substantially continuously supplying a stream of an alkaline metal hydroxide selected from the group consisting of lithium hydroxide, sodium hydroxide, potassium hydroxide, and rubidium hydroxide to the reaction vessel.

4. The continuous process of claim 1, wherein substantially continuously supplying a stream of an alkaline metal hydroxide or an alkaline metal alkoxide to the reaction vessel comprises substantially continuously supplying a stream of an alkaline metal isopropoxide selected from the group consisting of lithium isopropoxide, sodium isopropoxide, potassium isopropoxide, rubidium isopropoxide, cesium isopropoxide and francium isopropoxide to the reaction vessel.

5. The continuous process of claim 1, further comprising increasing the temperature and pressure of the reaction vessel to above ambient temperature and pressure while substantially continuously mixing the nitroaromatic, the alkaline metal hydroxide or the alkaline metal alkoxide, and the one of methanol and isopropanol in the reaction vessel to produce the first mixture.

6. A continuous process for preparing 1-chloro-2,4-dinitroanisole, comprising:
substantially continuously supplying a stream of 1-chloro-2,4-dinitrobenzene to a reaction vessel;
substantially continuously supplying a stream of methanol to the reaction vessel;
substantially continuously supplying a stream of an alkaline metal hydroxide to the reaction vessel;
substantially continuously mixing the 1-chloro-2,4-dinitrobenzene, methanol and alkaline metal hydroxide in the reaction vessel to produce a first mixture;
stripping any unreacted methanol from the first mixture to produce a second mixture;
subjecting the second mixture to a countercurrent wash with water to produce a third mixture comprising water and 1-chloro-2,4-dinitroanisole; and
drying the third mixture to recover the 1-chloro-2,4-dinitroanisole.

7. The continuous process of claim 6, wherein substantially continuously supplying a stream of an alkaline metal hydroxide to the reaction vessel comprises substantially continuously supplying a stream of an alkaline metal hydroxide selected from the group consisting of lithium hydroxide, sodium hydroxide, potassium hydroxide, and rubidium hydroxide to the reaction vessel.

8. The continuous process of claim 6, further comprising increasing the temperature and pressure of the reaction vessel to above ambient temperature and pressure while substantially continuously mixing the 1-chloro-2,4-dinitrobenzene, methanol and alkaline metal hydroxide in the reaction vessel to produce the first mixture.

9. A continuous process for preparing 1-isopropoxy-2,4-dinitrobenzene, comprising:
substantially continuously supplying a stream of 1-chloro-2,4-dinitrobenzene to a reaction vessel;
substantially continuously supplying a stream of isopropanol to the reaction vessel;
substantially continuously supplying a stream of an alkaline metal alkoxide to the reaction vessel;
substantially continuously mixing the 1-chloro-2,4-dinitrobenzene, isopropanol and alkaline metal alkoxide in the reaction vessel to produce a first mixture;
stripping any unreacted isopropanol from the first mixture to produce a second mixture;
subjecting the second mixture to a countercurrent wash with water to produce a third mixture comprising water and 1-isopropoxy-2,4-dinitrobenzene; and
drying the third mixture to recover the 1-isopropoxy-2,4-dinitrobenzene.

10. The continuous process of claim 9, wherein substantially continuously supplying a stream of an alkaline metal alkoxide to the reaction vessel comprises substantially continuously supplying a stream of an alkaline metal isopropoxide selected from the group consisting of lithium isopropoxide, sodium isopropoxide, potassium isopropoxide, and rubidium isopropoxide.

11. The continuous process of claim 9, further comprising increasing the temperature and pressure of the reaction vessel to above ambient temperature and pressure while substantially continuously mixing the 1-chloro-2,4-dinitrobenzene, isopropanol and alkaline metal isopropoxide in the reaction vessel to produce the first mixture.

12. A continuous process for preparing alkoxynitroarenes, comprising:
substantially continuously supplying a stream of a nitroaromatic to a reaction vessel;
substantially continuously supplying a stream of an alcohol solvent to the reaction vessel, the alcohol solvent comprising between one and four carbon atoms that are a straight chain or a branched chain;
substantially continuously supplying a stream of an alkaline metal alkoxide to the reaction vessel;
substantially continuously mixing the nitroaromatic, the alcohol solvent, and the alkaline metal alkoxide in the reaction vessel to produce a first mixture;
stripping any unreacted alcohol solvent from the first mixture to produce a second mixture;
subjecting the second mixture to a countercurrent wash with water to produce a third mixture comprising water and the alkoxynitroarene; and
drying the third mixture to recover the alkoxynitroarene.

13. The method of claim 12, wherein substantially continuously supplying a stream of an alcohol solvent to the reaction vessel comprises substantially continuously supplying a stream of methanol or isopropanol to the reaction vessel.
